(19) United States
(12) Patent Application Publication
(10) Pub. No.: US 2025/0297312 A1
(43) Pub. Date: Sep. 25, 2025

(54) EPIGENETIC MARKERS FOR DETECTING OXIDATIVE STRESS

(71) Applicant: Evonik Operations GmbH, Essen (DE)

(72) Inventors: Florian BÖHL, Neckargemünd (DE); Suki ROY, Singapore (SG); Jennifer BOURLAND, Singapore (SG); Sanjanaa NAGARAJAN, Singapore (SG)

(73) Assignee: Evonik Operations GmbH, Essen (DE)

(21) Appl. No.: 18/862,218

(22) PCT Filed: Apr. 21, 2023
(86) PCT No.: PCT/EP2023/060485

§ 371 Date: Nov. 1, 2024

(30) Foreign Application Priority Data
May 3, 2022 (EP) 22171355.5

(51) Int. Cl. C12Q 1/6883

(52) U.S. Cl. CPC C12Q 1/6883; C12Q 2600/154

(57) ABSTRACT

The present invention is related to a method of identifying oxidative stress (OS) in a test cell, the method comprising:
(a) determining the methylation status of at least five genes in a DNA sample obtained from the test cell;
(b) comparing the methylation status of the genes from step (a) to the methylation status of the corresponding genes in a control without OS,
   wherein a difference in the methylation status of the genes in the test cell compared to the corresponding genes in the control is indicative of the cell having OS; and
   wherein the genes in step (a) are selected from the group consisting of PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9, AUTS2, and CACNA1C
   and the regulatory regions of the same.

### FIELD OF THE INVENTION

The present invention relates to a method for detecting oxidative stress (OS) in a cell. In particular, the method is capable of identifying OS in cell by using a gene panel with at least two genes that are differentially methylated in a cell with OS relative to a cell without OS. More in particular, the differential methylation takes place on in the gene body and the regulatory region of the genes in the gene panel.

### BACKGROUND OF THE INVENTION

Living organisms are subjected continuously to a variety of stresses from the outside environment. In order to resist such stresses, they maintain their homeostasis by various regulatory systems. Oxidative stress refers to a serious imbalance between the levels of reactive oxygen species (ROS) in a cell and its antioxidant defense mechanism. In order to survive this stress, living organisms have a system called redox regulation to cope with the stress to maintain their homeostasis by regulating the redox state. This system functions to adapt to many external stress agents such as radiation, ultraviolet (UV) light rays, environmental pollutants, high fever, low temperature, hypoxic condition, and infectious diseases as well as to oxidative stress from lifestyle-related diseases such as cancer, diabetes, arteriosclerosis, hypertension and obesity. However, if this regulation mechanism is broken for some reason or other, oxidative stress (OS) occurs. OS can lead to cellular damage, DNA fragmentation, apoptosis and cell death. Early detection of OS can prevent further damage in the living organism causing the organism to receive early treatment or start using protection.

There are some methods known in the art for early detection of OS. However, none of these methods known in the art have been officially used to detect OS in a cell by using a genomic sample of the body.

The human skin is constantly exposed to oxidative stress and free radicals, such as to high quantities of ROS, derived not only from ordinary metabolic reactions but also continuous exposure to air, radiation and UV light rays, environmental pollutants, as well as physical and/or chemical agents (e.g., cosmetics). Under some conditions, the production of ROS may become so great that is may contribute to the pathogenesis of, for example, psoriasis or skin cancer. Oxidative damage caused by free radicals such as ROS is also a main cause of physical ageing in general, and of the skin in particular. Accordingly, there is a need in the art for detection of OS in cells, for example skin cells to prevent further damage to the cells.

There are some methods known in the art for early detection of OS. However, none of these methods known in the art have been officially used to detect OS in a cell by using a genomic sample of the body.

The human skin is constantly exposed to oxidative stress and free radicals, such as to high quantities of ROS, derived not only from ordinary metabolic reactions but also continuous exposure to air, radiation and UV light rays, environmental pollutants, as well as physical and/or chemical agents (e.g., cosmetics). Under some conditions, the production of ROS may become so great that it may contribute to the pathogenesis of, for example, psoriasis or skin cancer. Oxidative damage caused by free radicals such as ROS is also a main cause of physical ageing in general, and of the skin in particular. Accordingly, there is a need in the art for early detection of OS in cells, for example skin cells to prevent further damage to the cells.

### DESCRIPTION OF THE INVENTION

The present invention attempts to solve the problems above by providing a method of using a gene panel with at least two genes that are differentially methylated in a cell with OS. In particular, at least two genes including Protein Tyrosine Phosphatase Receptor Type N2 (PTPRN2), differential methylation of which, is capable of being used for detecting OS in a cell.

Since environmental factors/agents such as, UV light exposure, ageing, diet and the like, may trigger OS which can further induce an alteration in the promoter CpG methylation status of the gene by recruiting DNA methyltransferases (DNMTs) and TET enzymes to various promoters, biomarkers that result in differential methylation in a cell with OS is essential to overcome the problems mentioned above. In particular, genes which can be used as biomarkers for detecting OS in a cell include PTPRN2 and other specific genes. PTPRN2 and other specific genes in a cell with OS are differentially methylated (i.e. hypomethylated or hypermethylated) compared to the corresponding genes in a cell without OS. Accordingly, PTPRN2 and other specific genes may be effectively used to determine if a cell has OS. Similarly, a gene panel comprising at least PTPRN2 and other specific genes may be used to detect OS in a cell as these genes will be differentially methylated compared to a cell without OS. This is particularly advantageous as using epigenetics provides a means of predicting the onset of OS in a cell, thus allowing OS to be treated earlier before causing even more damage to the cell. Further, an epigenetic marker is a long-term biomarker, that is to say it is inheritable and can be used to detect OS in the next generation as well if need be.

According to one aspect of the present invention, there is provided a method of identifying oxidative stress (OS) in a test cell, the method comprising:
(a) determining the methylation status of at least five genes in a DNA sample obtained from the test cell;
(b) comparing the methylation status of the genes from step (a) to the methylation status of the corresponding genes in a control without OS,
   wherein a difference in the methylation status of the genes in the test cell compared to the corresponding genes in the control is indicative of the cell having OS; and wherein the genes in step (a) are selected from the group consisting of PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9, AUTS2, CACNA1C,
   and the regulatory regions of the same.

As used herein, the term “cell” refers to an intact live cell, naturally occurring or modified. The cell may be isolated from other cells, mixed with other cells in a culture, or within a tissue (partial or intact), or an organism. In particular, the cell may be a eukaryote cell. More in particular, the cell may be mammalian cell. The term “mammalian cell” refers to any cell derived from a mammalian subject. The cell may also be a cell derived from the culture and expansion of a cell obtained from a subject. The cell may also have been genetically modified to express a recombinant protein and/or nucleic acid. The mammalian cell may be from humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In particular, the subject is a mammal. More in particular, the mammal is selected from the group consisting of a mouse, a rat, a guinea pig, a dog, a mini-pig, a human being, a cow, a sheep, a pig, a goat, a horse, a donkey, and a mule. In particular, the mammalian cell may be a skin cell, a stem cell or a cell derived therefrom. More in particular, the mammalian cell may be a skin cell.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in a cell with OS compared to a cell with no OS.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745). In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to a cell with no indication of OS.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample. In particular, control refers to a cell with no indication of OS.

As used herein, the term “gene” refers to the respective genomic DNA sequence, including any promoter and regulatory sequences of the gene (e.g., enhancers and other gene sequences involved in regulating expression of the gene), and/or the body of the gene in itself. A gene sequence may be an expressed sequence (e.g., expressed RNA, mRNA, cDNA). Further, where SNPs are known within genes the term shall be taken to include all sequence variants thereof.

As used herein, the term “genomic material” refers to nucleic acid molecules or fragments of the genome of the subject or group of subjects. In particular, such nucleic acid molecules or fragments are DNA or RNA or hybrids thereof, and most preferably are molecules of the DNA genome of a subject or group of subjects.

As used herein, the “promoter” or “gene promoter” used interchangeably with the term ‘regulatory region’ or ‘regulatory sequence’ refers to the respective contiguous gene DNA sequence extending from 1.5 kb upstream to 1.5 kb downstream relative to the transcription start site (TSS), or contiguous portions thereof. In particular, ‘regulatory region’ refers to the respective contiguous gene DNA sequence extending from 1.5 kb upstream to 0.5 kb downstream relative to the TSS. In some examples, ‘regulatory region’ refers to the respective contiguous gene DNA sequence extending from 1.5 kb upstream to the downstream edge of a CpG island that overlaps with the region from 1.5 kb upstream to 1.5 kb downstream from TSS (and is such cases, my thus extend even further beyond 1.5 kb downstream), and contiguous portions thereof. In particular, with respect to PTPRN2, any CpG dinucleotide of the gene that is coordinately methylated with the ‘regulatory region’ of the gene, has substantial diagnostic/classification utility as disclosed herein.

As used herein, the “DNA sample” refers to the DNA extracted from the cell according to any aspect of the present invention using known methods in the art.

In particular, when there is differential methylation detected in a test cell, that is to say that the cell displays hypermethylation or hypomethylation at, at least one CpG site in comparison to the control (i.e., a cell without indication of OS), then the test cell has OS.

In particular, in the method according to any aspect of the present invention, in step (a) the methylation status of at least 1, 2, 3, 4, 5, 6, 7, 8, 9, 10, 11, 12, 13, 14, 15, 16, or 17, 18, 19, 20, 21, 22, 23 or 24 genes are determined. More in particular, in step (a) the methylation status of at least 5, 6, 7, 8, 9, 10, 11, 12, 13, 14, 15, 16, or 17, 18, 19, 20, 21, 22, 23, 24 or25 genes are determined.

In one example, the methylation status of at least 5 genes are determined in step (a). The 5 genes are PTPRN2, MAD1L1, PRDM16, TNXB, and HDAC4.

In another example, the methylation of at least 6 genes are determined in step (a). The 6 genes are PTPRN2, MAD1L1, PRDM16, TNXB, HDAC4, and ADARB2.

In another example, the methylation of at least 7 genes are determined in step (a). The 7 genes are PTPRN2, MAD1L1, PRDM16, TNXB, HDAC4, ADARB2, and CDH4.

In another example, the methylation of at least 8 genes are determined in step (a). The 8 genes are PTPRN2, MAD1L1, PRDM16, TNXB, HDAC4, ADARB2, CDH4, and DIP2C.

In another example, the methylation of at least 9 genes are determined in step (a). The 9 genes are PTPRN2, MAD1L1, PRDM16, TNXB, HDAC4, ADARB2, CDH4, DIP2, and SHANK2.

In another example, the methylation of at least 10 genes are determined in step (a). The 10 genes are PTPRN2, MAD1L1, PRDM16, TNXB, HDAC4, ADARB2, CDH4, DIP2, SHANK2, and CAMTA1.

In another example, the methylation of at least 11 genes are determined in step (a). The 11 genes are PTPRN2, MAD1L1, PRDM16, TNXB, HDAC4, ADARB2, CDH4, DIP2, SHANK2, CAMTA1, and RPTOR.

In another example, the methylation of at least 12 genes are determined in step (a). The 12 genes are PTPRN2, MAD1L1, PRDM16, TNXB, HDAC4, ADARB2, CDH4, DIP2, SHANK2, CAMTA1, RPTOR and RASA3.

In a further example, the methylation of at least 13 genes are determined in step (a). The 13 genes are PTPRN2, MAD1L1, PRDM16, TNXB, HDAC4, ADARB2, CDH4, DIP2, SHANK2, CAMTA1, RPTOR, RASA3 and SDK1.

In a further example, the methylation of at least 14 genes are determined in step (a). The 14 genes are PTPRN2, MAD1L1, PRDM16, TNXB, HDAC4, ADARB2, CDH4, DIP2, SHANK2, CAMTA1, RPTOR, RASA3, SDK1 and AGAP1.

In a further example, the methylation of at least 15 genes are determined in step (a). The 15 genes are PTPRN2, MAD1L1, PRDM16, TNXB, HDAC4, ADARB2, CDH4, DIP2C, SHANK2, CAMTA1, RPTOR, RASA3, SDK1, AGAP1 and TBCD.

In a further example, the methylation of at least 16 genes are determined in step (a). The 16 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD and SEPT9.

In a further example, the methylation of at least 17 genes are determined in step (a). The 17 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9 and FRMD4A.

In a further example, the methylation of at least 18 genes are determined in step (a). The 18 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A and MCF2L.

In a further example, the methylation of at least 19 genes are determined in step (a). The 19 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L and FOXP1.

In a further example, the methylation of at least 20 genes are determined in step (a). The 20 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1 and RPS6KA2.

In a further example, the methylation of at least 21 genes are determined in step (a). The 21 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2 and SORCS2.

In another example, the methylation of at least 22 genes are determined in step (a). The 22 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2 and NXN.

In another example, the methylation of at least 23 genes are determined in step (a). The 23 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN and TRAPPC9.

In another example, the methylation of at least 24 genes are determined in step (a). The 24 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9 and AUTS2.

In another example, the methylation of at least 25 genes are determined in step (a). The 25 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9, AUTS2 and CACNA1C. More in particular, one of the genes in step (a) is the gene Protein Tyrosine Phosphatase Receptor Type N2 (PTPRN2), and/or the regulatory region of PTPRN2. PTPRN2 is phosphatidylinositol phosphatase with the ability to dephosphorylate phosphatidylinositol 3-phosphate and phosphatidylinositol 4,5-diphosphate which plays important roles in lipid signaling, cell signaling and membrane trafficking. The differential methylation of the gene body and/or regulatory region of PTPRN2 is indicative of OS in the cell relative to a cell without OS. That is to say, the hypomethylation or hypermethylation of the PTPRN2, and/or the regulatory region of PTPRN2 is indicative of OS.

The genes in step (a) are selected from the group consisting of Adenosine Deaminase RNA Specific B2 (ADARB2), Nucleoredoxin (NXN), Sidekick Cell Adhesion Molecule 1 (SDK1), Calmodulin Binding Transcription Activator 1 (CAMTA1), MCF.2 Cell Line Derived Transforming Sequence Like (MCF2L), Sortilin Related VPS10 Domain Containing Receptor 2 (SORCS2), Disco Interacting Protein 2 Homolog C (D/P2C), FERM Domain Containing 4A (FRMD4A), histone deacetylase 4 (HDAC4), Mitotic spindle assembly checkpoint protein (MAD1L1), PR/SET Domain 16 (PRDM16), Ras GTPase-activating protein 3 (RASA3), Ribosomal Protein S6 Kinase A2 (RPS6KA2), Tubulin Folding Cofactor D (TBCD), tenascin-X (TNXB), Trafficking Protein Particle Complex Subunit 9 (TRAPPC9), and), ArfGAP With GTPase Domain (AGAP1), Calcium channel, voltage-dependent, L type, alpha 1C subunit (CACNA1C), Regulatory Associated Protein Of MTOR Complex 1 (RPTOR), Cadherin 4 (CDH4), SEPTIN9 (SEPT9), Forkhead Box P1 (FOXP1), Activator Of Transcription And Developmental Regulator (AUTS2) and SH3 And Multiple Ankyrin Repeat Domains 2 (SHANK2).

In one example, the genes in step (a) may also include the genes provided in Table 3.

The method according to any aspect of the present invention, further comprises the step of:
(i) performing bisulfite modification to the DNA sample before step (a).

‘Bisulfite treatment’ of genomic DNA used interchangeably with the term ‘bisulfite modification’, refers to the treatment of the genomic DNA with a deaminating agent such as a bisulfite that may be used to treat all DNA, methylated or not. In particular, the term “bisulfite” as used herein encompasses any suitable type of bisulfite, such as sodium bisulfite, or other chemical agents that are capable of chemically converting a cytosine (C) to an uracil (U) without chemically modifying a methylated cytosine and therefore can be used to differentially modify a DNA sequence based on the methylation status of the DNA, e.g., U.S. Pat. Pub. US 2010/0112595. As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

Accordingly, before step (a) according to any aspect of the present invention is carried out, the genomic DNA contained/obtained or extracted from the cell, is first bisulfite treated.

An alternative method available in the art may be used instead of bisulfite treatment. A skilled person will understand which other methods to use. In one example, TET-assisted pyridine borane sequencing (TAPS) may be used for detection of 5mC and 5hmC (Yibin Liu, et al., Nature Biotechnology, 37: 424-429 (2019).

The cell used according to any aspect of the present invention is obtained from a biological sample selected from the group consisting of blood, brain, sperm and any other tissue or sample that provides genomic DNA to be used in the method according to any aspect of the present invention.

In particular, the biological sample may comprise any biological material obtained from the subject that contains DNA, and may be liquid, solid or both, may be tissue or bone, or a body fluid such as blood, lymph, etc. In particular, the biological sample useful for the present invention may comprise biological cells or fragments thereof.

The term “test” used in conjunction with the term cell herein refers to a cell that is subjected to the method according to any aspect of the present invention and is the basis for an analysis application of the present invention. A ‘test cell’ is therefore a cell or a group of cells being tested according to any aspect of the present invention or a profile being obtained or generated in this context. Conversely, the term “reference” or ‘control’ shall denote, mostly predetermined, entities which are used for a comparison with the test entity. In particular, a ‘test cell’ refers to a cell being tested for OS where the methylation status has to be determined and a ‘control’ refers to a cell without OS where the methylation status is already known and used as a reference.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next. More in particular, the epigenetic marker for OS in a cell is the gene body and/or regulatory region of PTPRN2 and at least one other gene. The differential methylation of these genes in a test cell relative to a cell with no OS (i.e. the control cell) is indicative of OS in the test cell. In particular, promoter-specific hyper/hypomethylation leads to changes in the expression of different genes. More in particular, the differential methylation of PTPRN2 and the other genes result in the differential expression of PTPRN2. This differential expression of the gene relative to a control, where there is no differential methylation, is indicative of OS in the cell tested.

According to a further aspect of the present invention, there is provided a gene panel with at least five genes for detecting the presence of OS in a cell, wherein one of the five genes is PTPRN2 and the other four genes are selected from the group consisting of

MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9, AUTS2, and CACNA1C.

According to yet another aspect of the present invention, there is provided a use of a gene panel with at least five genes for indicating the presence of OS in a cell, wherein one of the five genes is PTPRN2 and the other four genes are selected from the group consisting of MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9, AUTS2, and CACNA1C

and wherein differential methylation of at least the five genes indicates the presence of OS in the cell.

In particular, the differential methylation refers to the hypermethylation or hypomethylation of the genes.

### BRIEF DESCRIPTION OF FIGURES

FIG. 1 is a graph showing the number of probes that were differentially methylated in different gene bodies and promoter regions of cells where artificial OS (high UV for 24 hrs) was induced according to Example 1. As can be seen, genes, MAD1L1 (meiotic spindle arrest component) and PTPRN2 (similar to receptor-like protein tyrosine phosphatases) have the highest differentially methylated probe distribution. The other genes which are also differentially methylated in a cell with OS are shown.

FIG. 2 is a graph showing the number of probes that were differentially methylated in different gene bodies and promoter regions of cells artificial OS (low UV for 72 hrs) was induced according to Example 1. As can be seen, genes, MAD1L1 (meiotic spindle arrest component) and PTPRN2 (similar to receptor-like protein tyrosine phosphatases) have the highest differentially methylated probe distribution. The other genes which are also differentially methylated in a cell with OS are shown.

FIG. 3 is a graph showing the number of probes that were differentially methylated in different gene bodies and promoter regions of cells where artificial OS (high H₂O₂ for 24 hrs) was induced according to Example 2. As can be seen, genes, MAD1L1 (meiotic spindle arrest component) and PTPRN2 (similar to receptor-like protein tyrosine phosphatases) have the highest differentially methylated probe distribution. The other genes which are also differentially methylated in a cell with OS are shown.

FIG. 4 is a graph showing the number of probes that were differentially methylated in different gene bodies and promoter regions of cells artificial OS (low H₂O₂ for 24 hrs) was induced according to Example 2. As can be seen, genes, MAD1L1 (meiotic spindle arrest component) and PTPRN2 (similar to receptor-like protein tyrosine phosphatases) have the highest differentially methylated probe distribution. The other genes which are also differentially methylated in a cell with OS are shown.

FIG. 5 is a graph showing the number of probes that were differentially methylated in different gene bodies and promoter regions of cells where artificial OS according to Example 3 with Medox® in cells was induced. As can be seen, genes, MAD1L1 (meiotic spindle arrest component) and PTPRN2 (similar to receptor-like protein tyrosine phosphatases) have the highest differentially methylated probe distribution. The other genes which are also differentially methylated in a cell with OS are shown.

FIG. 6 is a graph showing the top 20 overlapping genes at different treatments.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Oxidative Stress on Human Tissue with UV Light

Artificial oxidative stress was induced in the cell culture system and skin tissue model to analyze the methylation status of promoters.

T-Skin models were obtained from Episkin SA, France which is composed of reconstructed human skin. Each skin model consists of a dermal equivalent overlaid by a stratified, well-differentiated epidermis derived from normal human keratinocytes. Upon receiving the skin models, it was recovered by incubating in T-Skin culture medium overnight at 37° C. in a 5% CO₂ incubator.

To induce oxidative stress on a human tissue system, skin models (5× replicates) were exposed to UV radiation (UVA 24 J/cm²+UVB 50 mJ/cm²) (high UV) daily for 24 hrs.

In another group, skin models (5× replicates) were exposed to UV radiation (UVA 12 J/cm²+UVB 25 mJ/cm² daily) (low UV) daily for 72 hrs.

Exposure to UV radiation leads to the generation of ROS which finally results in the development of oxidative stress within the cells. A control set of skin models (5× replicates) were maintained for 72 hrs without any exposure to UV radiation. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

### Quality Control and Data Processing

Methylation EPIC array data processing was performed in R version 4.1.2 (2021 Nov. 1) using the minfi version 1.40.0. The raw intensity data (IDAT) were imported into the R (4.1.2), processed using the minfi (1.4.0) Bioconductor package, 18. Quality check on samples was performed to keep probes that have a detection P-value<0.01 in one or more samples or have a mean detection P-value<0.05 in all samples. Then samples were normalized using functional normalization (implemented by preprocessFunnorm function in minfi) for type-bias correction and background correction.

Prior to differential methylation analysis, the probes with non-specific binding, cross reactive probes, probes affected by common SNPs, and probes annotated to the X,Y chromosomes were also filtered out. Beta-value and M-value of normalized and filtered samples were calculated using getBeta and getM function respectively, the samples were then subjected to further downstream analysis.

### Differential Methylation Analysis

Differential methylation analysis was performed using packages limma version 3.50.1 and DMRcate version 2.8.5. Contrast matrix was set up by comparing each corresponding treatment and control group and empirical Bayesian algorithm was used to fit the M-values based on the design and contrast model. Probes with adjusted P-value lower than 0.05 were considered as differentially methylation positions (DMPs). Annotation was performed using IlluminaHumanMethylationEPICkanno.ilmn12.hg19 and annotatr package (1.20.0).

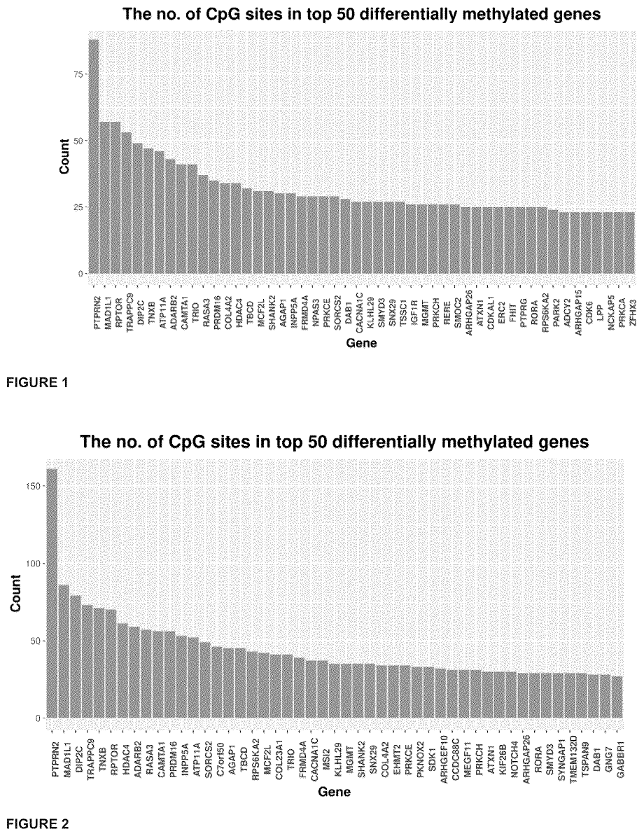

As seen in FIGS. 1 and 2, one of the top genes that were differentially methylated in the promoter region is PTPRN2 (Protein Tyrosine Phosphatase Receptor Type N2). The other genes that were also significantly differentially methylated is shown in FIGS. 1 and 2.

### Example 2

Oxidative Stress on Human Tissue with H₂O₂

Another way to induce oxidative stress on a human tissue system is through Hydrogen peroxide treatment which leads to the generation of ROS within the cells. The skin models (5× replicates) were treated with two different concentrations of Hydrogen peroxide [100 μM (low) and 200 μM (high)] for 2 hrs and were maintained for 24 hrs. A control set of skin models (5× replicates) were maintained for 24 hrs without any treatment with Hydrogen peroxide. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

Same method of quality control and data processing as that disclosed in Example 1 was carried out on the samples here. Further, the same differential methylation analysis as disclosed in Example 1 was carried out on the data obtained from Example 2.

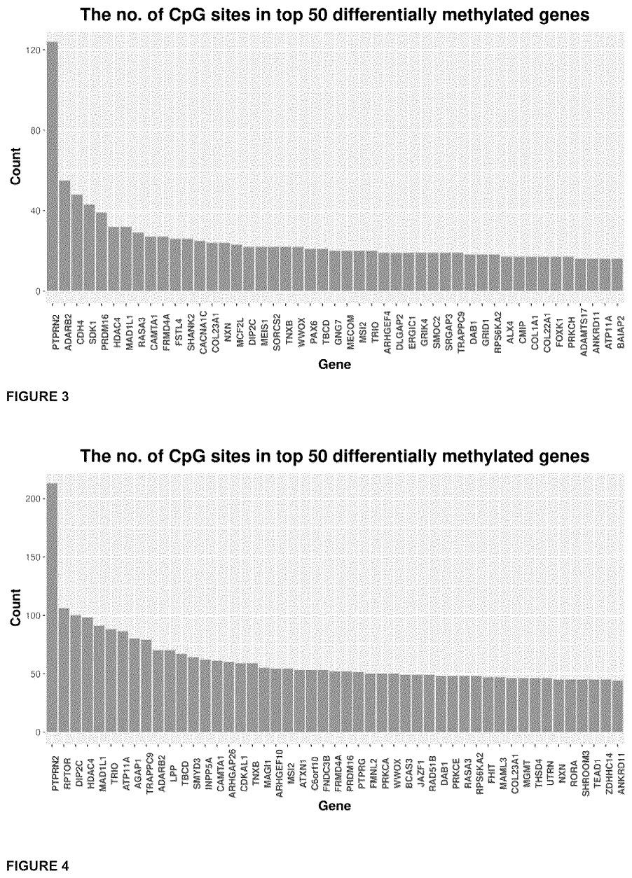

The results are shown in FIGS. 3 and 4.

### Example 3

Oxidative Stress on Cell Culture System with Medox®

To investigate oxidative stress in the cell culture system, Mesenchymal Stem Cells (MSCs) were treated with Medox® (Evonik, Batch:H-080719) which contains a lot of natural anthocyanins, associated with antioxidative and anti-inflammatory properties. Bone marrow derived MSCs were cultured for 1 week in Mesencult ACF Plus Medium with two doses of Medox® (4× replicates): 25 μg/ml (low) and 100 μg/ml (high). The media with Medox® was replaced every second day for 1 week. As a control (4× replicates), MSCs were cultured for 1 week in Mesencult ACF Plus Medium without any Medox® treatment. Medox® treatment is expected to produce the opposite reaction to OS.

This was followed by collection of cell pellet and genomic DNA was purified from the cell pellet using the DNeasy® Blood & Tissue Kit (Qiagen). The genomic DNA was quantified using the PicroGreen® or NanoDrop™ 2000.

The genomic DNA (500 ng) from the cell pellet was subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

DNA methylation profiling has been proven to be a powerful analytical tool to accurately identify the origin of tissue and the effect of environmental factors. It has several advantages as a biomarker classifier as it is a stable marker, and it can facilitate quantitative analysis at single-nucleotide resolution.

Same method of quality control and data processing as that disclosed in Example 1 was carried out on the samples here. Further, the same differential methylation analysis as disclosed in Example 1 was carried out on the data obtained from Example 3.

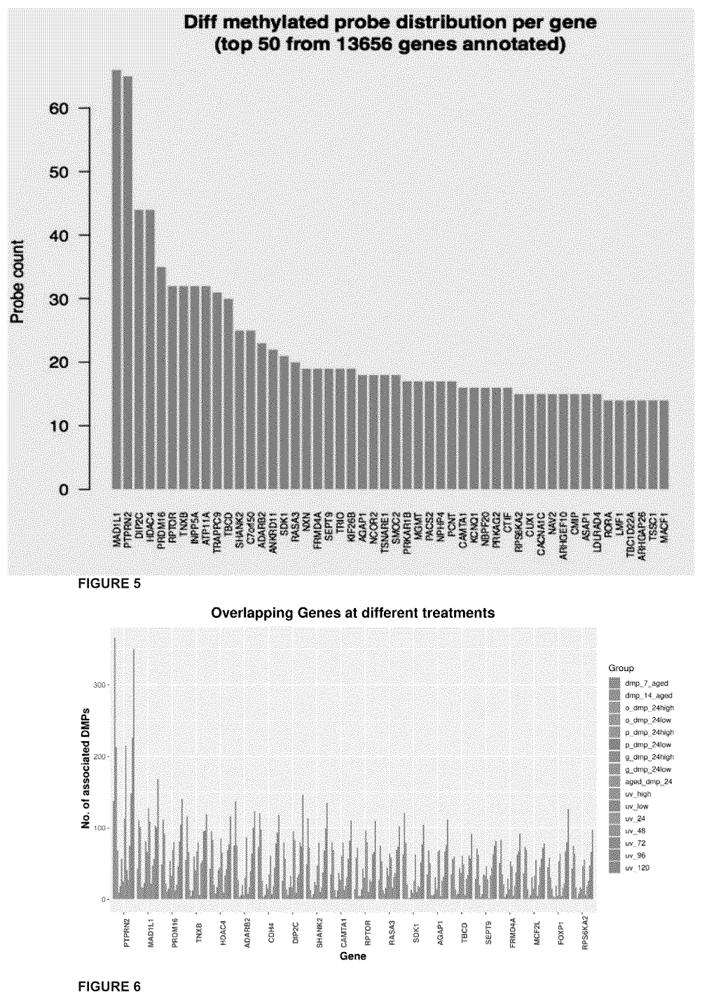

With the low treatment to Medox® on MSCs, 35,532 differentially methylated probes (p<0.05) were identified, out of which 15,368 probes were hypermethylated and 20,164 probes were hypomethylated. These probes were associated with 13,656 unique genes which are generally hypomethylated across promoter and gene body with up to 10-15% methylation difference as compared to the control group. One of the top 50 genes that was identified from the 13,656 unique genes was PTPRN2 (Protein Tyrosine Phosphatase Receptor Type N2) amongst other genes involved in Rho Kinase-mediated signalling, cell to cell junction, cell polarity and vesicle transport. This is shown in FIG. 5. In particular, the top candidates were associated with the Rho-GTPase pathways, the disc-large complex and histone-methyltransferase. The Rho pathway is required for stress fibre formation and focal adhesion (where the cytoskeleton/cell connects integrins with the ECM).

Results of all Examples (i.e. Examples 1, 2 and 3)

The genes that were significantly differentially methylated from Example 1 were juxtaposed with the genes that were significantly differentially methylated from Examples 2 and 3. The list of genes are shown in Table 1 below.

TABLE 1
Genes differentially methylated in the 5 categories: (i) high UV (Example 1), (ii) low UV
(Example 1), (iii) high H₂O₂ (Example 2), (iv) low H₂O₂ (Example 2) and (v) high Medox ® (Example 3).

| Genes | Genes | Genes | Genes | Genes | Genes | Genes |
|---|---|---|---|---|---|---|
| differentially | differentially | differentially | differentially | differentially | differentially | differentially |
| methylated | methylated | methylated | methylated | methylated | methylated | methylated |
| in 5 | in 4 | in 3 | in 2 | in 1 | in 1 | in 1 |
| categories | categories | categories | categories | category | category | category |
| ADARB2 | AGAP1 | ANKRD11 | ARHGEF10 | ADAMTS17 | COL22A1 | MACF1 |
| ATP11A | ARHGAP26 | KLHL29 | C7orf50 | ADCY2 | DLGAP2 | MAEA |
| CAMTA1 | ATXN1 | MCF2L | CDKAL1 | ALX4 | ERC2 | MAGI1 |
| DIP2C | CACNA1C | MSI2 | COL4A2 | ARHGAP15 | ERGIC1 | MAML3 |
| FRMD4A | COL23A1 | NXN | EHMT2 | ARHGEF10L | FMNL2 | MECOM |
| HDAC4 | DAB1 | PRKCE | FHIT | ARHGEF4 | FNDC3B | MEGF11 |
| MAD1L1 | INPP5A | PRKCH | GNG7 | BAIAP2 | FOXK1 | MEIS1 |
| PRDM16 | MGMT | RORA | KIF26B | BCAS3 | FSTL4 | MYT1L |
| PTPRN2 | RASA3 | SDK1 | LPP | BCR | GABBR1 | NCKAP5 |
| TBCD | RPS6KA2 | SMOC2 | PRKCA | C6orf10 | GAK | NOTCH4 |
| TNXB | RPTOR | SMYD3 | PTPRG | CCDC88C | GPR133 | NPAS3 |
| TRAPPC9 | SHANK2 | SORCS2 | SNX29 | CDH4 | GRID1 | OSBPL5 |
| TRIO | | | TSPAN9 | CDK6 | GRIK4 | PARK2 |
| | | | TSSC1 | CELSR1 | IGF1R | PAX6 |
| | | | WWOX | CMIP | ISPD | PCNT |
| | | | | COL1A1 | JAZF1 | PEX14 |
| | | | | PKNOX2 | SHROOM3 | SYNGAP1 |
| | | | | PRKAR1B | SLC12A7 | TEAD1 |
| | | | | RAD51B | SRGAP3 | THSD4 |
| | | | | RERE | SSBP3 | TMEM132D |
| | | | | TSNARE1 | UTRN | ZFHX3 |
| | | | | TTC7B | ZDHHC14 | ZMIZ1 |

The three independent experiment setups to analyze the methylation status of promoters during oxidative stress have shown differential methylation of PTPRN2 promoter and other genes, suggesting these genes to be a potential biomarker for oxidative stress.

### Example 4

Oxidative Stress on Human Tissue with UV Light Rays

Artificial oxidative stress was induced in the cell culture system and skin tissue model to analyze the methylation status of promoters.

T-Skin models were obtained from Episkin SA, France which is composed of reconstructed human skin. Each skin model consists of a dermal equivalent overlaid by a stratified, well-differentiated epidermis derived from normal human keratinocytes. Upon receiving the skin models, it was recovered by incubating in T-Skin culture medium overnight at 37° C. in a 5% CO₂ incubator.

To induce oxidative stress on a human tissue system, skin models (5× replicates) were exposed to UV radiation (UVA 24 J/cm²+UVB 50 mJ/cm²) (high UV) and cultured for 24 hrs.

In another group, skin models (5× replicates) were exposed to UV radiation (UVA 12 J/cm²+UVB 25 mJ/cm² daily) (low UV) and cultured for 24 hrs.

Exposure to UV radiation leads to the generation of ROS which finally results in the development of oxidative stress within the cells. A control set of skin models (5× replicates) were maintained for 24 hrs without any exposure to UV radiation. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

Oxidative Stress on Human Tissue with H₂O₂

Another way to induce oxidative stress on a human tissue system is through Hydrogen peroxide treatment which leads to the generation of ROS within the cells. The skin models (5× replicates) were treated with two different concentrations of Hydrogen peroxide [100 μM (low) and 200 μM (high)] for 2 hrs and were maintained for 24 hrs. A control set of skin models (5× replicates) were maintained for 24 hrs without any treatment with Hydrogen peroxide. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

Oxidative Stress on Human Tissue with Particulate Matter 2.5

Another way to induce oxidative stress on a human tissue system is through Particulate Matter 2.5 (PM2.5) treatment which leads to the generation of ROS within the cells by its chemical components and metals. The skin models (5× replicates) were treated with two different concentrations of PM2.5 [15 μg/cm² (low) and 30 μg/cm² (high)] and were maintained for 24 hrs. A control set of skin models (5× replicates) were maintained for 24 hrs without any treatment with PM2.5. Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

Oxidative Stress on Human Tissue with Glyoxal

Another way to induce oxidative stress on a human tissue system is through Glyoxal treatment which provokes oxidative stress by increasing the level of ROS within the cells by producing advanced glycation end-products. The skin models (5× replicates) were treated with two different concentrations of glyoxal [0.5 mM (low) and 1 mM (high)] and were maintained for 24 hrs. A control set of skin models (5× replicates) were maintained for 24 hrs without any treatment with glyoxal.

Followed by the treatment, skin models were collected, and genomic DNA was purified from the tissue samples using the DNeasy® Blood & Tissue Kit (Qiagen). The genomic DNA was quantified using the PicroGreen® or NanoDrop™ 2000.

The genomic DNA (500 ng) from tissue samples were subjected to bisulfite conversion using the EZ DNA Methylation-Gold™ Kit (Zymo Research). The methylation levels were quantified using Infinium MethylationEPIC v2.0 Kit (Illumina) which can analyze over 850,000 methylation sites quantitatively across the genome at single-nucleotide resolution.

Oxidative Stress on Human Tissue with Ageing

Another way to induce oxidative stress on a human tissue system is through ageing which leads to the generation of Reactive oxygen and nitrogen species (RONS) within the cells.

The skin models were maintained in the deep well plate with media for 7 days (4× replicates), 14 days (4× replicates) and 21 days (4× replicates) respectively to induce ageing in the skin tissue. Skin models were collected after 7 days, 14 days and 21 days respectively, and genomic DNA was purified from the tissue samples using the DNeasy® Blood & Tissue Kit (Qiagen). The genomic DNA was quantified using the PicroGreen® or NanoDrop™ 2000.

### Quality Control and Data Processing

A total of 103 samples from 16 different treatments and their respective controls were analyzed. The treatments were grouped into two batches. Batch 1 consisted of all 24 hour treatments, at high and low concentrations. All treatments in batch 1 shared the same control group. Table 2 shows the sample information for batch 1.

TABLE 2
Batch 1 samples

| Group | Treatment | Replicates |
|---|---|---|
| Control | Untreated, 24 h | 5 |
| Pollution Treatment | Treated with 30 μg/cm² particulate | 5 |
| | matter 2.5 for 24 h | |
| | Treated with 15 μg/cm² particulate | 5 |
| | matter 2.5 for 24 h | |
| H₂O₂Treatment | Treated with systemic | 5 |
| | exposure to 200 μM H₂O₂ for | |
| | 2 h and cultured for 24 h | |
| | Treated with systemic | 5 |
| | exposure to 100 μM H₂O₂ for | |
| | 2 h and cultured for 24 h | |
| Glyoxal Treatment | Treated with systemic | 5 |
| | exposure to 1 mM glyoxal 1 | |
| | mM for 24 h | |
| | Treated with systemic exposure | 5 |
| | to 0.5 mM glyoxal 1 | |
| | mM for 24 h | |
| UV-radiation | Exposed to UVA 24 J/cm2 + | 5 |
| Treatment | UVB 50 mJ/cm2 and | |
| | cultured for 24 h | |
| | Exposed to UVA 12 J/cm2 + | 5 |
| | UVB 25 mJ/cm2and | |
| | cultured for 24 h | |
| Aged | Untreated, cultured for | 6 |
| | 2 weeks in media | |

Methylation EPIC array data processing was performed in R version 4.2.2 (2021 Nov. 10 r83330) using the minfi version 1.42.0. The raw intensity data (IDAT) were imported into R (4.2.2) and processed using the minfi (1.42.0) Bioconductor package. Quality check on samples were performed to keep probes that had a detection P-value<0.01 in one or more samples or had a mean detection P-value<0.05 in all samples. The samples were then normalized using functional normalization (implemented by preprocesssFunnorm function in minfi) for type-bias correction and background correction.

Prior to differential methylation analysis, the probes with non-specific binding, cross reactive probes, probes affected by common SNPs, and probes annotated to the X,Y chromosomes were also filtered out. Beta-value and M-value of normalized and filtered samples were calculated using getBeta and getM function respectively, the samples were then subjected to further downstream analysis.

### Differential Methylation Analysis

Pair-wise differential methylation analysis (total of 16 pairs) was performed using the limma package version 3.52.4. The batch 1 samples were analyzed together, while for the batch 2 samples, the UV light and aged samples were analyzed separately. Contrast matrix was set up by comparing each corresponding treatment and control group and empirical Bayesian algorithm was used to fit the M-values based on the design and contrast model. Probes with adjusted P-value lower than 0.05 were considered as differentially methylation positions (DMPs). Annotation was performed using IlluminaHumanMethylationEPICanno.ilm10b2.hg19. Genes that overlapped at all treatments were then identified. As seen in Table 3, there are a total of 1250 genes in common between the 16 comparisons. The rows indicate the total number of DMPs that appear for that gene in each comparison. FIG. 6 shows the top 20 genes seen in all 16 comparisons.

TABLE 3
List of 1250 genes that are differentially
methylated in all treatments

1. PTPRN2
2. MAD1L1
3. PRDM16
4. TNXB
5. HDAC4
6. ADARB2
7. CDH4
8. DIP2C
9. SHANK2
10. CAMTA1
11. RPTOR
12. RASA3
13. SDK1
14. AGAP1
15. TBCD
16. SEPT9
17. FRMD4A
18. MCF2L
19. FOXP1
20. RPS6KA2
21. SORCS2
22. NXN
23. TRAPPC9
24. AUTS2
25. CACNA1C
26. PDE4D
27. PRKCZ
28. ANKRD11
29. CMIP
30. NCOR2
31. INPP5A
32. KCNQ1
33. COL23A1
34. C7orf50
35. ABR
36. NTM
37. ATP11A
38. SNX29
39. TRIO
40. CBFA2T3
41. NAV2
42. LPP
43. ZMIZ1
44. CUX1
45. CASZ1
46. KLHL29
47. PITX2
48. HIVEP3
49. PARD3
50. RORA
51. PRKAR1B
52. RERE
53. HCCA2
54. GALNT9
55. FSTL4
56. MECOM
57. PRKCA
58. TBC1D22A
59. DPP6
60. PPP2R2C
61. LRP5
62. SH3RF3
63. SMYD3
64. SH3PXD2A
65. SYN3
66. MAGI1
67. TACC2
68. RGS12
69. ARID1B
70. DAB1
71. ATXN1
72. PRKAG2
73. DLGAP2
74. GNG7
75. ARHGEF10
76. RPH3AL
77. TP73
78. GRID1
79. KCNMA1
80. CTBP2
81. NFATC1
82. FOXN3
83. SLIT3
84. ADAMTS2
85. ZFHX3
86. MBP
87. TSNARE1
88. CCDC88C
89. CDH23
90. TCF4
91. SMG6
92. PLEC1
93. THSD4
94. PKNOX2
95. KDM4B
96. MGMT
97. SRGAP3
98. C9orf3
99. MICAL3
100. GMDS
101. SORBS2
102. PCSK6
103. ANKS1B
104. IGF1R
105. CELSR1
106. NOTCH4
107. ZBTB20
108. NEDD4L
109. SYNE2
110. ANO1
111. KIF26B
112. RUNX1
113. RAI1
114. SLC12A7
115. B3GNTL1
116. SLCO3A1
117. FGFR2
118. AFAP1
119. LMF1
120. IQSEC1
121. SDK2
122. EBF3
123. PCDHGA4
124. LRP1
125. MACROD2
126. TSSC1
127. EXT1
128. MAST4
129. SKI
130. KIRREL3
131. MACROD1
132. HRNBP3
133. MEGF11
134. NRG1
135. CACNA1H
136. TIAM1
137. SHROOM3
138. THADA
139. ERGIC1
140. EXOC4
141. PARK2
142. VGLL4
143. DNAH17
144. NFIX
145. PHACTR1
146. ROBO1
147. ASAP1
148. GLI2
149. RIMBP2
150. DLGAP1
151. ERC2
152. LTBP1
153. IFT140
154. SNTG2
155. EHMT1
156. KIAA0182
157. MEGF6
158. TEAD1
159. COL11A2
160. FARP1
161. CTNNA2
162. KCNAB2
163. MAGI2
164. PACRG
165. PDE4DIP
166. HOXC4
167. RNF220
168. NRXN1
169. GRK5
170. HECW1
171. AHRR
172. CAPZB
173. FAM53B
174. KNDC1
175. SND1
176. GLIS1
177. ODZ4
178. PCCA
179. ITPK1
180. LDLRAD4
181. VAV2
182. ZC3H3
183. TSPAN9
184. CDKAL1
185. BACH2
186. OBSCN
187. SLC38A10
188. TTC7B
189. CREB5
190. CTIF
191. TRIM26
192. CDH22
193. PDZD2
194. C10orf11
195. COL22A1
196. ABTB2
197. CCNY
198. MYO18A
199. ACOT7
200. DDR1
201. KDM2B
202. PRDM2
203. SH3BP4
204. COL5A1
205. GPC6
206. PTPRG
207. ESRRG
208. JARID2
209. CORO2B
210. MAN1C1
211. ATP2B2
212. MAML2
213. LRBA
214. MACF1
215. PACS2
216. ASAP2
217. DLGAP4
218. PLEKHG5
219. ARHGEF10L
220. ARHGAP22
221. MICAL2
222. SIPA1L3
223. COL18A1
224. EPHB2
225. GAK
226. PAX5
227. CTBP1
228. PTPRF
229. RXRA
230. TBC1D16
231. BLCAP
232. DENND1A
233. MSRA
234. NPAS3
235. IGSF21
236. PC
237. RGMA
238. RGS6
239. BCAR3
240. SYNJ2
241. BCL2
242. GPR133
243. SIM1
244. LHFPL2
245. VAC14
246. AFF3
247. ARHGEF3
248. BAHCC1
249. FHOD3
250. NCALD
251. SFMBT2
252. INPP4B
253. PLEKHA6
254. CDK6
255. COBL
256. GLIS3
257. LRRFIP1
258. KCNN3
259. TNRC18
260. ATP10A
261. FAM19A5
262. GTF2IRD1
263. KSR1
264. BTBD9
265. CDC42BPB
266. RTN4RL1
267. MAPK8IP3
268. SPEG
269. BRSK2
270. LARGE
271. PRKG1
272. AP2A2
273. ARHGAP10
274. ARHGEF7
275. FHAD1
276. SAMD12
277. SETBP1
278. GALNT2
279. PXDN
280. GTDC1
281. HK1
282. PTPN14
283. FAM38A
284. PLXNA4
285. CLEC16A
286. ERG
287. GRM4
288. LINC00299
289. PTPRS
290. RAP1GAP2
291. SPTBN1
292. STK10
293. ADAP1
294. PLCH2
295. SLC9A3
296. COL13A1
297. PITPNC1
298. RIN3
299. TRPM1
300. FMN1
301. MRC2
302. ZFYVE28
303. OCA2
304. OSBP2
305. SBNO2
306. STK24
307. MPRIP
308. PARVB
309. PLEKHA7
310. ANK2
311. BRE
312. FAM20C
313. FAM83A
314. GNAS
315. NRG2
316. BCAR1
317. PAX7
318. PDZRN3
319. SLC43A2
320. ZEB2
321. MEIS2
322. MTUS1
323. PTK2B
324. TBC1D1
325. CDK14
326. GABRG3
327. PCGF3
328. PDE2A
329. TCERG1L
330. TRAK1
331. MYO10
332. ZFPM1
333. ACSF3
334. CADM1
335. MEF2C
336. MYO1E
337. PLCG2
338. JPH3
339. SORBS1
340. AACS
341. EIF2C2
342. RAB40C
343. MTSS1
344. SVIL
345. ADGRG1
346. CLMN
347. INTS1
348. PFKP
349. RBMS1
350. NOS1AP
351. SEMA6A
352. AATK
353. AGAP3
354. PDE1C
355. CHD7
356. HPCAL1
357. LGR6
358. SPIDR
359. CELF2
360. KAZN
361. VPS13B
362. VTI1A
363. DGKZ
364. LOC105376360
365. PDE11A
366. PFKFB3
367. POU6F2
368. FHL2
369. JAKMIP3
370. MIB2
371. RABGAP1L
372. AHNAK
373. BAT2
374. CTNND2
375. DENND3
376. EHMT2
377. RGS7
378. RUNX2
379. TOLLIP
380. VOPP1
381. EYA2
382. HIPK2
383. LIMS1
384. PVRL1
385. RASGRF1
386. VIPR2
387. FAM189A1
388. LDB2
389. CARD14
390. ITGB4
391. NR3C1
392. PITPNM2
393. C6orf10
394. COLEC11
395. KLF12
396. NOTCH1
397. PEMT
398. LTBP3
399. MARK2
400. NTN1
401. PTPRE
402. TOX2
403. COL27A1
404. DLG5
405. FANCC
406. ZNF407
407. DDAH1
408. KIAA1949
409. MTHFD1L
410. PTPRK
411. ACOX3
412. BNC2
413. DLX5
414. NPHP4
415. BRD2
416. CNTNAP2
417. CRYL1
418. DNAJB6
419. HSPG2
420. NCOA2
421. FAM178B
422. ARHGEF17
423. FAM65C
424. KIF26A
425. RBM19
426. RIN2
427. CHCHD6
428. KCNQ5
429. TNK2
430. MAEA
431. MCTP1
432. MGRN1
433. RNF213
434. DIP2B
435. FAM160A1
436. GFOD1
437. PKD1
438. ARNT2
439. RBFOX3
440. SYNGAP1
441. CACNB4
442. FMN2
443. GATAD2A
444. RYR1
445. SRPK2
446. CCDC57
447. COL9A3
448. DOT1L
449. TMEM132C
450. TTC7A
451. CABIN1
452. VEPH1
453. ARID3A
454. CD82
455. MAPK4
456. PLXNB2
457. SGK1
458. DGKD
459. WNT3
460. CSGALNACT1
461. CYFIP1
462. ETS1
463. PKP3
464. WWC1
465. ZNF365
466. ZNF827
467. A2BP1
468. GNAO1
469. MGLL
470. SCARB1
471. SH3BP2
472. GRID2
473. PPM1H
474. SRCIN1
475. ST7
476. TFDP1
477. ADARB1
478. DAPK1
479. FLJ22536
480. ROR2
481. VAX2
482. CACNA2D4
483. CACNB2
484. CDYL
485. AFAP1L2
486. ELL
487. OXR1
488. RBPMS
489. SPOCK1
490. EXD3
491. KIAA1522
492. KIFC3
493. OSBPL3
494. SPG7
495. UNC5C
496. ADCY5
497. FAT1
498. GFRA1
499. NRXN2
500. PLEKHG3
501. SEC14L1
502. CGNL1
503. CLASP1
504. PXN
505. SLC7A5
506. MYO7A
507. SBF2
508. TLL2
509. ASPH
510. CABLES1
511. CERS6
512. DYNC1H1
513. LSAMP
514. NLGN1
515. PIK3AP1
516. SULF2
517. TERT
518. ADAMTS16
519. AGRN
520. LARP1
521. MAP2
522. PLXND1
523. STK39
524. ARSB
525. HOXA3
526. PMEPA1
527. SPTBN4
528. CACNA1A
529. DNAH10
530. FAT3
531. INSR
532. PHF21B
533. SLC16A3
534. SLC24A4
535. ST3GAL2
536. TLN2
537. TNFAIP8
538. BMPR1B
539. CLSTN1
540. DNAH2
541. GNAQ
542. GREB1
543. PRRT1
544. ZNF385B
545. HMGA2
546. MEST
547. ODZ2
548. PIGG
549. ACTN4
550. ADPRHL1
551. C15orf27
552. FAM83F
553. HMCN1
554. HRH1
555. NRCAM
556. DPYD
557. FRMD4B
558. KIAA0513
559. KIF5C
560. PITPNM3
561. PLCL2
562. RAP1GAP
563. WDR27
564. XKR4
565. ATP2C1
566. CLIP2
567. DGKI
568. GALNTL6
569. GRIK3
570. JAK1
571. RGL2
572. SCFD2
573. TAPBP
574. VARS
575. GHR
576. ALPK2
577. CHID1
578. TGIF1
579. ISPD
580. NINJ2
581. SLC6A6
582. SLC9A3R2
583. SPTBN2
584. CUGBP2
585. OLFM2
586. RARA
587. RBM33
588. TULP4
589. AGPAT3
590. CACNA2D1
591. MEG3
592. PLB1
593. PRR5L
594. ZNF385A
595. AKAP12
596. HMHA1
597. L3MBTL4
598. SYNPO
599. BBX
600. HSF4
601. ITGB5
602. LTBP4
603. MIR5095
604. MKLN1
605. ZNRF1
606. BAI1
607. DPP9
608. EML6
609. ERBB4
610. PTPRD
611. RNF150
612. SERGEF
613. TJP2
614. ACTN1
615. BRUNOL4
616. EMX2OS
617. KLHDC4
618. MYO9B
619. TMEM51
620. TTC15
621. ABHD2
622. CHD3
623. EIF4G1
624. P4HB
625. PLA2G6
626. AHDC1
627. ANO6
628. ATP9B
629. CTNNBL1
630. IFFO2
631. INF2
632. NFATC4
633. SLC22A18
634. BCR
635. EPHA4
636. GGT1
637. SLC41A3
638. SYT7
639. UXS1
640. WDFY2
641. CHD9
642. ITGA6
643. KIAA1530
644. MIR99AHG
645. SMAD6
646. TNNT3
647. TXNRD2
648. UBR4
649. WDR90
650. ZBTB22
651. ZNF710
652. BAIAP3
653. KIAA1549
654. LSP1
655. USP6NL
656. FUT8
657. SNCAIP
658. DHRS3
659. RIMS1
660. TECPR2
661. TPCN1
662. ATM
663. NR5A2
664. NRP1
665. ROBO2
666. SHANK1
667. CACHD1
668. FSIP1
669. GRIA4
670. HCN4
671. MAP3K9
672. CTNNA1
673. PGS1
674. RFX4
675. STAG1
676. UBE2E1
677. WT1
678. CDH2
679. CUEDC1
680. RAMP1
681. ATG7
682. EDIL3
683. EPS8
684. MED27
685. MIR3134
686. RFX2
687. TBX2
688. TNFRSF19
689. TRIM27
690. CAMK4
691. FURIN
692. IGF2R
693. PTPN3
694. WNT5A
695. LRRC8D
696. PLXDC1
697. PPP1R13L
698. RCAN1
699. SORCS1
700. ADAMTS9
701. FBLIM1
702. FOXP4
703. GFPT2
704. IRAK2
705. NKD2
706. SRGAP1
707. TAOK3
708. TSC22D1
709. TSPAN4
710. ISM1
711. PHRF1
712. RHBDD1
713. TTC38
714. ZBTB46
715. FBXL18
716. MAP2K5
717. PLAGL1
718. PPFIA3
719. TRAM2
720. FBXO34
721. GABBR2
722. PAPLN
723. PNPLA6
724. SH2D3C
725. ST6GAL2
726. ABL1
727. EVI5L
728. PPFIA4
729. SACS
730. TBX1
731. TGFBI
732. TPCN2
733. ADSSL1
734. LOC645323
735. NEURL4
736. PPARG
737. TTBK1
738. ZEB1
739. GULP1
740. KCNQ2
741. LRP8
742. STK19
743. CDH5
744. CHN1
745. D2HGDH
746. DCC
747. IRF2
748. KIAA1462
749. PRDM8
750. RTN1
751. SLC10A7
752. TEF
753. ZSWIM4
754. CCDC40
755. COL12A1
756. EDARADD
757. ENTPD1
758. EVL
759. KCNC1
760. LOXL3
761. SMTN
762. SULT2B1
763. EDNRB
764. ERBB2
765. INS-IGF2
766. MLLT3
767. SLAIN1
768. TBC1D8
769. TCF25
770. TMC8
771. AKT2
772. C9orf171
773. CASR
774. FGFR3
775. FRMD6-AS2
776. MICA
777. MICALL2
778. MIPOL1
779. SMAD7
780. TESC
781. TMEM117
782. ZFP41
783. BDH1
784. BLK
785. GRK6
786. GTF2F2
787. HM13
788. ISLR2
789. NCAM2
790. PAK1
791. SCTR
792. THBS2
793. DNAH1
794. DPYS
795. FAM167A
796. GSK3B
797. LINC01599
798. PDLIM1
799. PPARD
800. SSU72
801. LAMB3
802. MYO7B
803. NXPH1
804. PSMB8
805. SNTG1
806. TRIOBP
807. UBE2D3
808. AMBRA1
809. FMNL3
810. HDAC7
811. HSPA12B
812. KIAA1688
813. RAB2A
814. RELL1
815. RHBDF2
816. SNX24
817. SOLH
818. ZMYM2
819. FLII
820. HIVEP1
821. KANSL1
822. LRTOMT
823. TFEB
824. TSPAN5
825. DNAJC17
826. HTRA1
827. QSOX1
828. SPSB4
829. TJP1
830. TPST2
831. TTLL10
832. AGPAT1
833. CDKN1C
835. GRASP
836. MAP1B
837. UBE2J2
838. USP2
839. ADAM22
840. ATOH8
841. DNAJC6
842. FAM171A1
843. H19
844. HLA-E
845. KCNK9
846. NKAIN3
847. SLC25A13
848. UNC13A
849. USP47
850. ZNF598
851. ADCY3
852. COL15A1
853. DAZAP1
854. DEDD2
855. ESPNP
856. FAM129B
857. LAMB1
858. LHX2
859. NDRG1
860. NKAIN2
861. PI4KA
862. PROM1
863. SLC27A1
864. CDK19
865. LAMC1
866. LRPAP1
867. NCS1
868. PDE10A
869. PPM1L
870. WNT3A
871. ZBTB4
872. ADAMTS18
873. APLP2
874. ASPG
875. BAZ2B
876. BSG
877. SDC1
878. SEMA4A
879. SGMS2
880. SPON1
881. TK2
882. ASB2
883. FLJ12825
884. GNA11
885. RBM38
886. SRRM4
887. ZBTB7B
888. ARHGEF18
889. BEGAIN
890. CXXC5
891. DAB2
892. DLG1
893. FAM110B
894. SLC17A7
895. TRABD
896. UBR3
897. WDTC1
898. CCDC88B
899. FGR
900. HLA-DOA
901. MAPK15
902. NXPH4
903. PRR16
904. SARDH
905. STK40
906. ACSL6
907. APC2
908. C10orf90
909. FOXF1
910. KCNH8
911. LEF1
912. PIK3R5
913. PITPNA
914. TELO2
915. YPEL2
916. ANO7
917. FBXW8
918. CYB561
919. ENG
920. FBLN5
921. NFAT5
922. POLR3E
923. SCN8A
924. SPATS2
925. TYK2
926. ZNF787
927. CCND3
928. CDC42BPA
929. FBXO42
930. KCNN1
931. LMO3
932. LPCAT3
933. MEGF8
934. SDCBP2
935. TGFA
936. ABCA1
937. ATP9A
938. EFCAB6
939. IDUA
940. KCNH1
941. NTNG2
942. PARP8
943. COL19A1
944. CPAMD8
945. FBN3
946. H2AFY
947. LINC00598
948. AMFR
949. AMOTL2
950. FAM120B
951. FAM83C
952. GRIN1
953. MNT
954. ABI2
955. BMF
956. HAND1
957. NECAB3
958. RNU5E
959. SATB1
960. SYT6
961. TBX4
962. ANP32A
963. C1orf94
964. CD63
965. CXCR5
966. GNB1L
967. GRIK5
968. UBXN11
969. UGP2
970. ADIPOR2
971. AK8
972. CBLC
973. HIP1R
974. HLF
975. PANX1
976. SLC1A4
977. SPNS2
978. TACC3
979. TMEM181
980. ZNF331
981. CCDC109B
982. CLDN10
983. FAM124A
984. LPPR1
985. LYSMD2
986. SIRPA
987. ST8SIA1
988. BAT3
989. CASP9
990. CHSY3
991. GPRC5C
992. KIAA0895L
993. 4-Sep
994. SETD3
995. SLC8A2
996. TMEM120B
997. ZNF503
998. AP2A1
999. CPM
1000. POLR1A
1001. VAT1L
1002. ZDHHC20
1003. CDC42BPG
1004. CPEB4
1005. CRAT
1006. ETV1
1007. EYA3
1008. IGFBP7
1009. LINGO3
1010. PLEKHG4B
1011. RAB7A
1012. REEP6
1013. SHC1
1014. TRPC7
1015. BRD4
1016. DHX34
1017. DNAH12
1018. FAM174B
1019. IGF2BP1
1020. SYNGR1
1021. APPL2
1022. C7orf49
1023. DUSP6
1024. MEGF10
1025. PRKAR1A
1026. SPINT1
1027. TMEM229B
1028. YIF1B
1029. ACVR2A
1030. AKAP1
1031. BZW2
1032. CLIP4
1033. DAGLB
1034. ERAP1
1035. EXT2
1036. KIF3B
1037. LRRC8A
1038. STK11
1039. SYT9
1040. TSHR
1041. VWA1
1042. ATP8B3
1043. CYGB
1044. E2F5
1045. MFAP2
1046. RHOT2
1047. STAMBPL1
1048. HS3ST4
1049. MAP3K4
1050. PEAR1
1051. RHOD
1052. 8-Sep
1053. TBX3
1054. B4GALT7
1055. BCL7C
1056. CCDC50
1057. CDC27
1058. CHD4
1059. FBXL16
1060. NLRC5
1061. PLA2G4F
1062. PYGB
1063. CDT1
1064. DPF1
1065. KL
1066. MAPK8IP1
1067. MFI2
1068. RARG
1069. WNT11
1070. ZNF638
1071. CTSD
1072. MSRB3
1073. SDCBP
1074. UBXN6
1075. DNAJC11
1076. ELP3
1077. GBF1
1078. PRR3
1079. SHC2
1080. SLC16A12
1081. UBE2L6
1082. EVC2
1083. OLFM3
1084. SLC17A9
1085. SLC5A2
1086. CABP1
1087. DTD1
1088. PDE6A
1089. ACAN
1090. B4GALT5
1091. C6orf132
1092. CUL9
1093. F7
1094. ITGA3
1095. KCNG2
1096. PLTP
1097. SEMA3B
1098. SNAP91
1099. STXBP5L
1100. ZBTB17
1101. C10orf105
1102. C11orf80
1103. FLYWCH1
1104. HOMER3
1105. PTPN21
1106. RASGRP2
1107. RER1
1108. RNF11
1109. RNPS1
1110. TXNDC5
1111. ALDOA
1112. IFRD1
1113. KLF10
1114. KLK5
1115. LY6G5C
1116. MYOCD
1117. OCIAD2
1118. PPP1R12C
1119. RNF4
1120. BAHD1
1121. BMP6
1122. CCDC64B
1123. FAM69A
1124. MATN1
1125. TMEM190
1126. ALDH16A1
1127. MIR548G
1128. MUPCDH
1129. RABEP1
1130. SLC29A3
1131. ARHGAP20
1132. CAND2
1133. CEACAM16
1134. CHDH
1135. FGF3
1136. LPAR2
1137. ORAI2
1138. SLC22A15
1139. STXBP1
1140. ZDHHC17
1141. ARL4D
1142. FLJ43860
1143. IQGAP3
1144. MST1P2
1145. WFS1
1146. ZCCHC2
1147. ABCC10
1148. ACVR1C
1149. MIER2
1150. XPC
1151. ZDHHC18
1152. BAD
1153. C5orf45
1154. DNASE1L3
1155. 11-Mar
1156. UBE2Q1
1157. WNT2
1158. ZNF507
1159. ZNF589
1160. C19orf25
1161. CEP68
1162. CYHR1
1163. GABRR2
1164. SCARB2
1165. SSH3
1166. YWHAZ
1167. B2M
1168. DDX41
1169. HIC2
1170. HYAL1
1171. NCL
1172. PANK2
1173. PHKG2
1174. ELL3
1175. GABRA2
1176. PAK2
1177. PRSS8
1178. SIRT2
1179. TRNP1
1180. ANKRD27
1181. DCK
1182. GPR157
1183. KCTD15
1184. LINC00426
1185. MTF1
1186. PM20D1
1187. DUPD1
1188. TP53I3
1189. BPTF
1190. CCDC48
1191. ERGIC2
1192. GAS8
1193. NOBOX
1194. PRSS12
1195. SLC3A2
1196. ARHGEF26
1197. FAAH
1198. KIAA0564
1199. PCAT29
1200. VPS26B
1201. C8orf76
1202. LSM14A
1203. LSM4
1204. NOL12
1205. PRNP
1206. S100PBP
1207. HCK
1208. MUC1
1209. ZNF248
1210. PTGS1
1211. SLC2A10
1212. SNAP29
1213. ACR
1214. ANG
1215. MAPK12
1216. SMCO4
1217. ASB13
1218. BCAT2
1219. C1orf105
1220. C5orf38
1221. DLAT
1222. FAM131C
1223. GNB2L1
1224. ACOT13
1225. CDKN1A
1226. CYB5A
1227. PFN2
1228. UBE2Z
1229. VPS18
1230. DDX5
1231. G6PC3
1232. MIF
1233. MTHFD2
1234. BRMS1L
1235. CALHM1
1236. CD200
1237. KCNJ2
1238. RG9MTD2
1239. RGPD1
1240. TTC21A
1241. ZDHHC24
1242. HSPA5
1243. DBP
1244. LRRC75A-AS1
1245. NDUFA7
1246. COPB2
1247. WDR69
1248. OSTM1
1249. LGALS17A
1250. C10orf82

1-14. (canceled)

15. A method of identifying oxidative stress (OS) in a test cell, the method comprising:
(a) determining the methylation status of at least five genes in a DNA sample obtained from the test cell;
(b) comparing the methylation status of the genes from step (a) to the methylation status of the corresponding genes in a control without OS,
wherein a difference in the methylation status of the genes in the test cell compared to the corresponding genes in the control is indicative of the cell having OS; and wherein the 5 genes are PTPRN2, MAD1L1, PRDM16, TNXB, and HDAC4 and the regulatory regions of the same.

16. The method according to claim 15, wherein the methylation of at least 20 genes is determined.

17. The method according to claim 16, wherein the 20 genes are PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, and RPS6KA2.

18. The method according to claim 15, wherein in step (a) the methylation status of all the genes PTPRN2, MAD1L1, PRDM16, TNXB, HDAC4, ADARB2, CDH4, DIP2C, SHANK2, CAMTA1, RPTOR, RASA3, SDK1, AGAP1, TBCD, SEPT9, FRMD4A, MCF2L, FOXP1, RPS6KA2, SORCS2, NXN, TRAPPC9, AUTS2, and CACNA1C is determined.

19. The method according to claim 15, wherein the OS is brought about by ageing, Ultraviolet (UV) light exposure and/or H₂O₂ exposure.

20. The method according to claim 15, further comprising the step of:
(i) performing bisulfite modification to the DNA sample before step (a).

21. The method according to claim 15 wherein the cell is a eukaryote.

22. The method according to claim 15, wherein the cell is from a mammal.

23. The method according to claim 22, wherein the mammal is a mouse, a rat, a guinea pig, a dog, a mini-pig, a human being, a cow, a sheep, a pig, a goat, a horse, a donkey, and a mule.

24. The method according to claim 15, wherein the cell is a skin cell, a stem cell or a cell derived therefrom.
